(19) United States
(12) Patent Application Publication
(10) Pub. No.: US 2008/0214845 A1
(43) Pub. Date: Sep. 4, 2008

(54) ANTIBACTERIAL 6'-MODIFIED 4,5-SUBSTITUTED AMINOGLYCOSIDE ANALOGS

(75) Inventors: Michael T. Migawa, Carlsbad, CA (US); Xiaojing Wang, Foster City, CA (US); Eric Swayze, Carlsbad, CA (US); Richard H. Griffey, Vista, CA (US)

Correspondence Address: SEED INTELLECTUAL PROPERTY LAW GROUP PLLC, 701 FIFTH AVE, SUITE 5400, SEATTLE, WA, 98104, US

(73) Assignee: ISIS PHARMACEUTICALS, INC., Carlsbad, CA (US)

(21) Appl. No.: 12/040,615

(22) Filed: Feb. 29, 2008

Related U.S. Application Data
(63) Continuation of application No. PCT/US2006/034216, filed on Aug. 31, 2006.
(60) Provisional application No. 60/713,600, filed on Sep. 1, 2005.

(51) Int. Cl. C07D 315/00

(52) U.S. Cl. 549/415

(57) ABSTRACT

The present invention is directed to analogs of aminoglycoside compounds as well as their preparation and use as prophylactic or therapeutics against microbial infection.

### CROSS-REFERENCE TO RELATED APPLICATIONS

This application is a continuation of International PCT Patent Application No. PCT/US2006/034216, which was filed on Aug. 31, 2006, now pending, which claims the benefit under 35 U.S.C. § 119(e) of U.S. Provisional Patent Application No. 60/713,600 filed Sep. 1, 2005. These applications are incorporated herein by reference in their entireties.

### FIELD OF THE INVENTION

The present invention is directed to novel aminoglycoside compounds and synthetic methods for their preparation and use as therapeutic or prophylactic agents.

### BACKGROUND OF THE INVENTION

A particular interest in modern drug discovery is the development of novel low molecular weight orally-bioavailable drugs that work by binding to RNA. RNA, which serves as a messenger between DNA and proteins, was thought to be an entirely flexible molecule without significant structural complexity. Recent studies have revealed a surprising intricacy in RNA structure. RNA has a structural complexity rivaling proteins, rather than simple motifs like DNA. Genome sequencing reveals both the sequences of the proteins and the mRNAs that encode them. Since proteins are synthesized using an RNA template, such proteins can be inhibited by preventing their production in the first place by interfering with the translation of the mRNA. Since both proteins and the RNAs are potential drug targeting sites, the number of targets revealed from genome sequencing efforts is effectively doubled. These observations unlock a new world of opportunities for the pharmaceutical industry to target RNA with small molecules.

Classical drug discovery has focused on proteins as targets for intervention. Proteins can be extremely difficult to isolate and purify in the appropriate form for use in assays for drug screening. Many proteins require post-translational modifications that occur only in specific cell types under specific conditions. Proteins fold into globular domains with hydrophobic cores and hydrophilic and charged groups on the surface. Multiple subunits frequently form complexes, which may be required for a valid drug screen. Membrane proteins usually need to be embedded in a membrane to retain their proper shape. The smallest practical unit of a protein that can be used in drug screening is a globular domain. The notion of removing a single alpha helix or turn of a beta sheet and using it in a drug screen is not practical, since only the intact protein may have the appropriate 3-dimensional shape for drug binding. Preparation of biologically active proteins for screening is a major limitation in classical high throughput screening. Quite often the limiting reagent in high throughput screening efforts is a biologically active form of a protein which can also be quite expensive.

For screening to discover compounds that bind RNA targets, the classic approaches used for proteins can be superceded with new approaches. All RNAs are essentially equivalent in their solubility, ease of synthesis or use in assays. The physical properties of RNAs are independent of the protein they encode. They may be readily prepared in large quantity through either chemical or enzymatic synthesis and are not extensively modified in vivo. With RNA, the smallest practical unit for drug binding is the functional subdomain. A functional subdomain in RNA is a fragment that, when removed from the larger RNA and studied in isolation, retains its biologically relevant shape and protein or RNA-binding properties. The size and composition of RNA functional subdomains make them accessible by enzymatic or chemical synthesis. The structural biology community has developed significant experience in identification of functional RNA subdomains in order to facilitate structural studies by techniques such as NMR spectroscopy. For example, small analogs of the decoding region of 16S rRNA (the A-site) have been identified as containing only the essential region, and have been shown to bind antibiotics in the same fashion as the intact ribosome.

The binding sites on RNA are hydrophilic and relatively open as compared to proteins. The potential for small molecule recognition based on shape is enhanced by the deformability of RNA. The binding of molecules to specific RNA targets can be determined by global conformation and the distribution of charged, aromatic, and hydrogen bonding groups off of a relatively rigid scaffold. Properly placed positive charges are believed to be important, since long-range electrostatic interactions can be used to steer molecules into a binding pocket with the proper orientation. In structures where nucleobases are exposed, stacking interactions with aromatic functional groups may contribute to the binding interaction. The major groove of RNA provides many sites for specific hydrogen bonding with a ligand. These include the aromatic N7 nitrogen atoms of adenosine and guanosine, the O4 and O6 oxygen atoms of uridine and guanosine, and the amines of adenosine and cytidine. The rich structural and sequence diversity of RNA suggests to us that ligands can be created with high affinity and specificity for their target.

Although our understanding of RNA structure and folding, as well as the modes in which RNA is recognized by other ligands, is far from being comprehensive, significant progress has been made in the last decade (Chow, C. S.; Bogdan, F. M., Chem. Rev., 1997, 97, 1489, Wallis, M. G.; Schroeder, R., Prog. Biophys. Molec. Biol. 1997, 67, 141). Despite the central role RNA plays in the replication of bacteria, drugs that target these pivotal RNA sites of these pathogens are scarce. The increasing problem of bacterial resistance to antibiotics makes the search for novel RNA binders of crucial importance.

Certain small molecules can bind and block essential functions of RNA. Examples of such molecules include the aminoglycoside antibiotics and drugs such as erythromycin which binds to bacterial rRNA and releases peptidyl-tRNA and mRNA. Aminoglycoside antibiotics have long been known to bind RNA. They exert their antibacterial effects by binding to specific target sites in the bacterial ribosome. For the structurally related antibiotics neamine, ribostamycin, neomycin B, and paromomycin, the binding site has been localized to the A-site of the prokaryotic 16S ribosomal decoding region RNA (Moazed, D.; Noller, H. F., Nature, 1987, 327, 389). Binding of aminoglycosides to this RNA target interferes with the fidelity of mRNA translation and results in miscoding and truncation, leading ultimately to bacterial cell death (Alper, P. B.; Hendrix, M.; Sears, P.; Wong, C., J. Am. Chem. Soc., 1998, 120, 1965).

There is a need in the art for new chemical entities that work against bacteria with broad-spectrum activity. Perhaps the biggest challenge in discovering RNA-binding antibacterial drugs is identifying vital structures common to bacteria that can be disabled by small molecule drug binding. A challenge in targeting RNA with small molecules is to develop a chemical strategy which recognizes specific shapes of RNA. There are three sets of data that provide hints on how to do this: natural protein interactions with RNA, natural product antibiotics that bind RNA, and man-made RNAs (aptamers) that bind proteins and other molecules. Each data set, however, provides different insights to the problem.

Several classes of drugs obtained from natural sources have been shown to work by binding to RNA or RNA/protein complexes. These include three different structural classes of antibiotics: thiostreptone, the aminoglycoside family and the macrolide family of antibiotics. These examples provide powerful clues to how small molecules and targets might be selected. Nature has selected RNA targets in the ribosome, one of the most ancient and conserved targets in bacteria. Since antibacterial drugs are desired to be potent and have broad-spectrum activity these ancient processes fundamental to all bacterial life represent attractive targets. The closer we get to ancient conserved functions the more likely we are to find broadly conserved RNA shapes. It is important to also consider the shape of the equivalent structure in humans, since bacteria were unlikely to have considered the therapeutic index of their RNAs while evolving them.

A large number of natural antibiotics exist, these include the aminoglycosides, kirromycin, neomycin, paromomycin, thiostrepton, and many others. They are very potent, bactericidal compounds that bind RNA of the small ribosomal subunit. The bactericidal action is mediated by binding to the bacterial RNA in a fashion that leads to misreading of the genetic code. Misreading of the code during translation of integral membrane proteins is thought to produce abnormal proteins that compromise the barrier properties of the bacterial membrane.

Antibiotics are chemical substances produced by various species of microorganisms (bacteria, fungi, actinomycetes) that suppress the growth of other microorganisms and may eventually destroy them. However, common usage often extends the term antibiotics to include synthetic antibacterial agents, such as the sulfonamides, and quinolines, that are not products of microbes. The number of antibiotics that have been identified now extends into the hundreds, and many of these have been developed to the stage where they are of value in the therapy of infectious diseases. Antibiotics differ markedly in physical, chemical, and pharmacological properties, antibacterial spectra, and mechanisms of action. In recent years, knowledge of molecular mechanisms of bacterial, fungal, and viral replication has greatly facilitated rational development of compounds that can interfere with the life cycles of these microorganisms.

At least 30% of all hospitalized patients now receive one or more courses of therapy with antibiotics, and millions of potentially fatal infections have been cured. At the same time, these pharmaceutical agents have become among the most misused of those available to the practicing physician. One result of widespread use of antimicrobial agents has been the emergence of antibiotic-resistant pathogens, which in turn has created an ever-increasing need for new drugs. Many of these agents have also contributed significantly to the rising costs of medical care.

When the antimicrobial activity of a new agent is first tested a pattern of sensitivity and resistance is usually defined. Unfortunately, this spectrum of activity can subsequently change to a remarkable degree, because microorganisms have evolved the array of ingenious alterations discussed above that allow them to survive in the presence of antibiotics. The mechanism of drug resistance varies from microorganism to microorganism and from drug to drug.

The development of resistance to antibiotics usually involves a stable genetic change, heritable from generation to generation. Any of the mechanisms that result in alteration of bacterial genetic composition can operate. While mutation is frequently the cause, resistance to antimicrobial agents may be acquired through transfer of genetic material from one bacterium to another by transduction, transformation or conjugation.

For the foregoing reasons, there is a need for new chemical entities that possess antimicrobial activity. Further, in order to accelerate the drug discovery process, new methods for synthesizing aminoglycoside antibiotics are needed to provide an array of compounds that are potentially new drugs for the treatment microbial infections.

### BRIEF SUMMARY OF THE INVENTION

In one aspect, the present invention provides compounds having formula I:

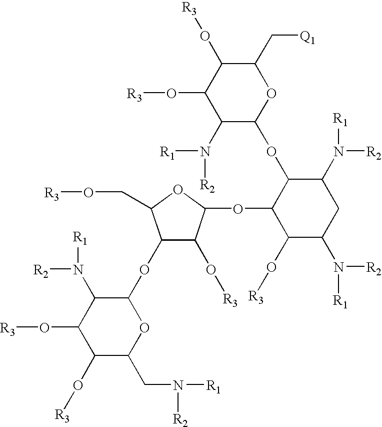

wherein:

each R₁ and R₂ is, independently, H or an amino protecting group;

each R₃ is, independently, H or a hydroxyl protecting group;

Q₁ is a NR₄R₅, a protected amino group or a nitrogen containing heterocycle wherein the heterocycle is covalently linked to the compound through the nitrogen atom;

R₄ is H, an amino protecting group, C₁-C₁₂ alkyl or substituted C₁-C₁₂ alkyl;

R₅ is amino, substituted amino, an amino protecting group, hydroxy, C₁-C₁₂ alkyl, substituted C₁-C₁₂ alkyl, C₂-C₁₂ alkenyl, substituted C₂-C₁₂ alkenyl, C₂-C₁₂ alkynyl or substituted C₂-C₁₂ alkynyl;

wherein the substituted groups are mono or poly substituted with optionally protected substituent groups each independently selected from halogen, OJ₃, NJ₁J₂, C(═O)—NJ₁J₂, N(H)C(═O)-J₁, N(J₁)-(CH₂)ₙ—OJ₃, N(J₁)-(CH₂)ₙ—NJ₁J₂, C₅-C₂₀ aryl, substituted C₅-C₂₀ aryl, C₅-C₇ alicyclic radical, substituted C₅-C₇ alicyclic radical, heterocycle radical, substituted heterocycle radical, azido, carboxy, acyl (C(═O)—X), ═O, cyano, sulfonyl (S(═O)₂—X) and sulfoxyl (S(═O)—X);
each X is, independently, H, C₁-C₁₂ alkyl or substituted C₁-C₁₂ alkyl;
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—X), substituted acyl, a heterocycle radical or a substituted heterocycle radical;
each J₃ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ aminoalkyl, substituted C₁-C₁₂ aminoalkyl or a hydroxyl protecting group; and
n is from 1 to 20.

In one embodiment each R₁ and R₂ is H. In another embodiment each R₃ is H. In another embodiment each R₁ and R₂ is H and each R₃ is H. In a further embodiment each of the substituent groups is independently, OH, NH₂, N(H)alkyl, C(═O)—N(H)J₂, N(H)C(═O)-J₁, N(J₁)-(CH₂)ₙ—OJ₃, N(J₁)-(CH₂)ₙ—NJ₁J₂, substituted C₅-C₇ alicyclic radical, C₅-C₇ alicyclic radical, C₅-C₂₀ aryl, substituted C₅-C₂₀ aryl, a heterocycle radical or a substituted heterocycle radical.

In one embodiment Q₁ is NR₄R₅. In another embodiment Q₁ is NR₄R₅ and R₄ is C₁-C₁₂ alkyl or substituted C₁-C₁₂ alkyl. In a further embodiment Q₁ is NR₄R₅ and R₄ is H. In another embodiment Q₁ is NR₄R₅ and R₅ is NH₂, C₁-C₁₂ alkyl or mono or poly substituted C₁-C₁₂ alkyl.

In one embodiment Q₁ is NR₄R₅ and R₅ is mono or poly substituted C₁-C₁₂ alkyl wherein each substituent group is independently selected from halogen, OH, NJ₁J₂, C₅-C₂₀ aryl, substituted C₅-C₂₀ aryl, C₅-C₇ alicyclic radical, substituted C₅-C₇ alicyclic radical, heterocycle radical and substituted heterocycle radical.

In another preferred embodiment Q₁ is NR₄R₅ and R₅ is mono or poly substituted C₁-C₁₂ alkyl wherein each substituent group is, independently, NH₂, phenyl, substituted phenyl, heterocycle radical or substituted heterocycle radical.

In another preferred embodiment Q₁ is NR₄R₅ and R₅ is mono or poly substituted C₁-C₁₂ alkyl wherein each substituent group is substituted phenyl wherein the substituted phenyl comprises at least one substituent group selected from halogen, C₁-C₁₂ alkyl, CF₃, alicyclic radical, OCH₃ and heterocyclic radical.

In another preferred embodiment Q₁ is NR₄R₅ and R₅ is a poly substituted C₁-C₁₂ alkyl comprising at least two substituent groups. In a preferred embodiment the two substituent groups are different and are selected from OH, C₁-C₁₂ alkyl, C₅-C₂₀ aryl and substituted C₅-C₂₀ aryl.

In another embodiment, wherein Q₁ is NR₄R₅ and R₄ is H, R₅ is not a C₁-C₁₂ alkyl or a monosubstituted C₁-C₁₂ alkyl wherein the substituent group is a C₅-C₂₀ aryl. In particular, in such an embodiment, R₅ is not a lower alkyl or a monosubstituted lower alkyl wherein the substituent group is a C₅-C₂₀ aryl.

In another embodiment, the compound of formula I is not IBIS00561141 or IBIS00561932.

The present invention also provides compounds that have specific stereochemistry about chiral centers having the configuration:

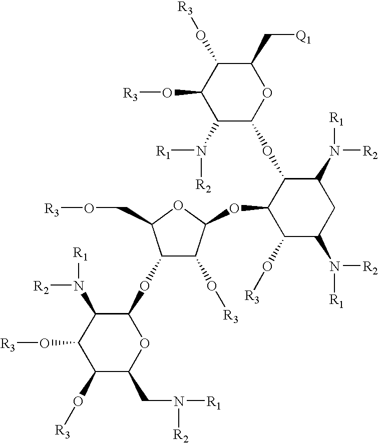

The present invention also prives methods of using a compound of the invention in therapy.

### DETAILED DESCRIPTION OF THE INVENTION

The present invention provides aminoglycoside compounds having formula I:

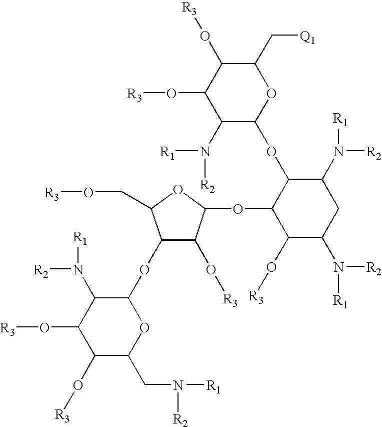

wherein:

each R₁ and R₂ is, independently, H or an amino protecting group;

each R₃ is, independently, H or a hydroxyl protecting group;

Q₁ is a NR₄R₅, a protected amino group or a nitrogen containing heterocycle wherein said heterocycle is covalently linked to said compound through the nitrogen atom;

R₄ is H, an amino protecting group, C₁-C₁₂ alkyl or substituted C₁-C₁₂ alkyl;

R₅ is amino, substituted amino, an amino protecting group, hydroxy, C₁-C₁₂ alkyl, substituted C₁-C₁₂ alkyl, C₂-C₁₂ alkenyl, substituted C₂-C₁₂ alkenyl, C₂-C₁₂ alkynyl or substituted C₂-C₁₂ alkynyl;

wherein said substituted groups are mono or poly substituted with optionally protected substituent groups each independently selected from halogen, OJ₃, NJ₁J₂, C(═O)—NJ₁J₂, N(H)C(═O)-J₁, N(J₁)-(CH₂)ₙ—OJ₃, N(J₁)-(CH₂)ₙ—NJ₁J₂, C₅-C₂₀ aryl, substituted C₅-C₂₀ aryl, C₅-C₇ alicyclic radical, substituted C₅-C₇ alicyclic radical, heterocycle radical, substituted heterocycle radical, azido, carboxy, acyl (C(═O)—X), ═O, cyano, sulfonyl (S(═O)₂—X) and sulfoxyl (S(═O)—X);
each X is, independently, H, C₁-C₁₂ alkyl or substituted C₁-C₁₂ alkyl;
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—X), substituted acyl, a heterocycle radical or a substituted heterocycle radical;
each J₃ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ aminoalkyl, substituted C₁-C₁₂ aminoalkyl or a hydroxyl protecting group; and
n is from 1 to 20.

In a preferred embodiment the compounds of the present invention are prepared from Paromomycin sulfate salt (commercially available from various sources including Sigma-Aldrich Co., et al.,). The reactive groups are orthogonally protected as illustrated in the examples below to prepare compounds of the invention. The methods disclosed herein are amenable to a wide variety of chemical reactions to prepare a large number of Paromomycin analogs. In some preferred embodiments of the present invention each R₁, R₂ and R₃ is H and Q₁ is substituted with a variety of functional groups.

The term “alkyl,” as used herein, refers to a saturated straight or branched hydrocarbon radical containing up to twenty four carbon atoms. Examples of alkyl groups include, but are not limited to, methyl, ethyl, propyl, butyl, isopropyl, n-hexyl, octyl, decyl, dodecyl and the like. Alkyl groups typically include from 1 to about 24 carbon atoms, more typically from 1 to about 12 carbon atoms (C₁-C₁₂ alkyl) with from 1 to about 6 carbon atoms being more preferred. The term “lower alkyl” as used herein includes from 1 to about 6 carbon atoms. Alkyl groups as used herein may optionally include one or more further substitutent groups (see substituent group list below).

The term “alkenyl,” as used herein, refers to a straight or branched hydrocarbon chain radical containing up to twenty four carbon atoms having at least one carbon-carbon double bond. Examples of alkenyl groups include, but are not limited to, ethenyl, propenyl, butenyl, 1-methyl-2-buten-1-yl, dienes such as 1,3-butadiene and the like. Alkenyl groups typically include from 2 to about 24 carbon atoms, more typically from 2 to about 12 carbon atoms with from 2 to about 6 carbon atoms being more preferred. Alkenyl groups as used herein may optionally include one or more further substitutent groups.

The term “alkynyl,” as used herein, refers to a straight or branched hydrocarbon radical containing up to twenty four carbon atoms and having at least one carbon-carbon triple bond. Examples of alkynyl groups include, but are not limited to, ethynyl, 1-propynyl, 1-butynyl, and the like. Alkynyl groups typically include from 2 to about 24 carbon atoms, more typically from 2 to about 12 carbon atoms with from 2 to about 6 carbon atoms being more preferred. Alkynyl groups as used herein may optionally include one or more further substitutent groups.

The term “aminoalkyl” as used herein, refers to an amino substituted alkyl radical. This term is meant to include C₁-C₁₂ alkyl groups having an amino substituent at any position and wherein the alkyl group attaches the aminoalkyl group to the parent molecule. The alkyl or amino portions of the aminoalkyl group can be further substituted with substituent groups.

The term “aliphatic,” as used herein, refers to a straight or branched hydrocarbon radical containing up to twenty four carbon atoms wherein the saturation between any two carbon atoms is a single, double or triple bond. An aliphatic group preferably contains from 1 to about 24 carbon atoms, more typically from 1 to about 12 carbon atoms with from 1 to about 6 carbon atoms being more preferred. The straight or branched chain of an aliphatic group may be interrupted with one or more heteroatoms that include nitrogen, oxygen, sulfur and phosphorus. Such aliphatic groups interrupted by heteroatoms include without limitation polyalkoxys, such as polyalkylene glycols, polyamines, and polyimines, for example. Aliphatic groups as used herein may optionally include further substitutent groups.

The term “alicyclic” refers to a cyclic ring system wherein the ring is aliphatic. The ring system can comprise one or more rings and wherein at least one ring is aliphatic. Alicyclics include rings having any degree of saturation. Preferred alicyclics include rings having from about 5 to about 9 carbon atoms in the ring. Alicyclic as used herein may optionally include further substitutent groups.

The term “alkoxy,” as used herein, refers to a radical formed between an alkyl group and an oxygen atom wherein the oxygen atom is used to attach the alkoxy group to a parent molecule. Examples of alkoxy groups include, but are not limited to, methoxy, ethoxy, propoxy, isopropoxy, n-butoxy, sec-butoxy, tert-butoxy, n-pentoxy, neopentoxy, n-hexoxy and the like. Alkoxy groups as used herein may optionally include further substitutent groups.

The terms “halo” and “halogen,” as used herein, refer to an atom selected from fluorine, chlorine, bromine and iodine.

The terms “aryl” and “aromatic,” as used herein, refer to a mono- or polycyclic carbocyclic ring system radicals having one or more aromatic rings. Examples of aryl groups include, but are not limited to, phenyl, naphthyl, tetrahydronaphthyl, indanyl, idenyl and the like. Preferred aryl ring systems have from about 5 to about 20 carbon atoms in one or more rings. Aryl groups as used herein may optionally include further substitutent groups.

The terms “aralkyl” and “arylalkyl,” as used herein, refer to a radical formed between an alkyl group and an aryl group wherein the alkyl group is used to attach the aralkyl group to a parent molecule. Examples include, but are not limited to, benzyl, phenethyl and the like. Aralkyl groups as used herein may optionally include further substitutent groups attached to the alkyl, the aryl or both groups that form the radical group.

The term “heterocyclic,” or “heterocyclic radical” as used herein, refers to a radical mono-, or poly-cyclic ring system that includes at least one heteroatom and is unsaturated, partially saturated or fully saturated, thereby including heteroaryl groups. Heterocyclic is also meant to include fused ring systems wherein one or more of the fused rings contain no heteroatoms. A heterocyclic group typically includes at least one atom selected from sulfur, nitrogen or oxygen. Examples of heterocyclic groups include, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, tetrahydrofuryl and the like. Heterocyclic groups as used herein may optionally include further substitutent groups.

The terms “heteroaryl,” and “heteroaromatic,” as used herein, refer to a radical comprising a mono- or poly-cyclic aromatic ring, ring system or fused ring system wherein at least one of the rings is aromatic and includes a heteroatom. Heteroaryl is also meant to include fused ring systems including systems where one or more of the fused rings contain no heteroatoms. Heteroaryl groups typically include one ring atom selected from sulfur, nitrogen or oxygen. Examples of heteroaryl groups include, but are not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like. Heteroaryl radicals can be attached to a parent molecule directly or through a linking moiety such as an aliphatic group or hetero atom. Heteroaryl groups as used herein may optionally include further substitutent groups.

The term “heteroarylalkyl,” as used herein, refers to a heteroaryl group as previously defined, attached to a parent molecule via an alkyl group. Examples include, but are not limited to, pyridinylmethyl, pyrimidinylethyl and the like. Heteroarylalkyl groups as used herein may optionally include further substitutent groups.

The term “acyl,” as used herein, refers to a radical formed by removal of a hydroxyl group from an organic acid and has the general formula —C(O)—X where X is typically aliphatic, alicyclic or aromatic. Examples include aliphatic carbonyls, aromatic carbonyls, aliphatic sulfonyls, aromatic sulfinyls, aliphatic sulfinyls, aromatic phosphates, aliphatic phosphates and the like. Acyl groups as used herein may optionally include further substitutent groups.

The terms “substituent and substituent group,” as used herein, are meant to include groups that are typically added to other groups or parent compounds to enhance desired properties or give desired effects. Substituent groups can be protected or unprotected and can be added to one available site or to many available sites in a parent compound. Substituent groups may also be further substituted with other substituent groups and may be attached directly or via a linking group such as an alkyl or hydrocarbyl group to the parent compound. Such groups include without limitation, halogen, hydroxyl, alkyl, alkenyl, alkynyl, acyl (—C(O)Rₐ), carboxyl (—C(O)O—Rₐ), aliphatic, alicyclic, alkoxy, substituted oxo (—O—Rₐ), aryl, aralkyl, heterocyclic, heteroaryl, heteroarylalkyl, amino (—NR_{b}R_{c}), imino(═NR_{b}), amido (—C(O)NR_{b}R_{c} or —N(R_{b})C(O)Rₐ), azido (—N₃), nitro (—NO₂), cyano (—CN), carbamido (—OC(O)NR_{b}R_{c} or —N(R_{b})C(O)ORₐ), ureido (—N(R_{b})C(O)NR_{b}R_{c}), thioureido (—N(R_{b})C(S)NR_{b}R_{c}), guanidinyl (—N(R_{b})C(═NR_{b})NR_{b}R_{c}), amidinyl (—C(═NR_{b})—NR_{b}R_{c} or —N(R_{b})C(NR_{b})Rₐ), thiol (—SR_{b}), sulfinyl (—S(O)R_{b}), sulfonyl (—S(O)₂R_{b}) and sulfonamidyl (—S(O)₂NR_{b}R_{c} or —N(R_{b})S(O)₂R_{b}). Wherein each Rₐ, R_{b} and R_{c} is a further substituent group with a preferred list including without limitation alkyl, alkenyl, alkynyl, aliphatic, alkoxy, acyl, aryl, aralkyl, heteroaryl, alicyclic, heterocyclic and heteroarylalkyl.

The term “protecting group,” as used herein, refers to a labile chemical moiety which is known in the art to protect reactive groups including without limitation, hydroxyl, amino and thiol groups, against undesired reactions during synthetic procedures. Protecting groups are typically used selectively and/or orthogonally to protect sites during reactions at other reactive sites and can then be removed to leave the unprotected group as is or available for further reactions. Protecting groups as known in the art are described generally in Greene and Wuts, Protective Groups in Organic Synthesis, 3rd edition, John Wiley & Sons, New York (1999).

Examples of hydroxyl protecting groups include, but are not limited to, benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl (BOC), isopropoxycarbonyl, diphenylmethoxycarbonyl, 2,2,2-trichloroethoxycarbonyl, 2-(tri-methylsilyl)ethoxycarbonyl, 2-furfuryloxycarbonyl, allyloxycarbonyl (Alloc), acetyl (Ac), formyl, chloroacetyl, trifluoroacetyl, methoxyacetyl, phenoxyacetyl, benzoyl (Bz), methyl, t-butyl, 2,2,2-trichloroethyl, 2-trimethylsilyl ethyl, 1,1-dimethyl-2-propenyl, 3-methyl-3-butenyl, allyl, benzyl (Bn), para-methoxybenzyldiphenylmethyl, triphenylmethyl (trityl), 4,4′-dimethoxytriphenylmethyl (DMT), substituted or unsubstituted 9-(9-phenyl)xanthenyl (pixyl), tetrahydrofuryl, methoxymethyl, methylthiomethyl, benzyloxymethyl, 2,2,2-trichloroethoxymethyl, 2-(trimethylsilyl)ethoxymethyl, methanesulfonyl, para-toluene-sulfonyl, trimethylsilyl, triethylsilyl, triisopropylsilyl, and the like. Preferred hydroxyl protecting groups for the present invention are DMT and substituted or unsubstituted pixyl.

Examples of amino protecting groups include, but are not limited to, t-butoxycarbonyl (BOC), 9-fluorenylmethoxycarbonyl (Fmoc), benzyloxycarbonyl, and the like.

Examples of thiol protecting groups include, but are not limited to, triphenylmethyl (Trt), benzyl (Bn), and the like.

Included within the scope of the present invention are the pharmaceutically acceptable salts of the foregoing compounds. As used herein, the term “pharmaceutically acceptable salts” refers to non-toxic acid addition salts and alkaline earth metal salts of the compounds of the invention. The salts can be prepared in situ during the final isolation and purification of the compounds of the invention, or separately by reacting the free base or acid functions with a suitable organic acid or base. Representative acid addition salts include the hydrochloride, hydrobromide, sulphate, bisulphate, acetate, oxalate, valerate, oleate, palmitate, stearate, laurate, borate, benzoate, lactate, phosphate, tosylate, mesylate, citrate, maleate, fumarate, succinate, tartrate, glucoheptonate, lactobionate, lauryl sulfate salts and the like. Representative alkali or alkaline earth metal salts include the sodium, calcium, potassium and magnesium salts.

### EXAMPLES

### General Procedures

¹H and ¹³C spectra were recorded on a 300 MHz and 75 MHz Bruker spectrometer, respectively. Silica gel 60 from EM Science was used for purification. The column for preparative LC-MS (Agilent) was a Luna C18 column (10 u, 250×21.20 mm) from Phenomenex. An isocratic gradient (1% AcOH in CH₃CN) was used as the mobile phase. All mass spectrometry data (API-ES) were obtained as a result of running the compounds through analytical LC-MS which simultaneously provided ELSD (Evaporative Light Scattering Detectors) and UV (Ultra-Visible at 254 nm) data.

### Example 1

### Preparation of N-Protected Paromomycin

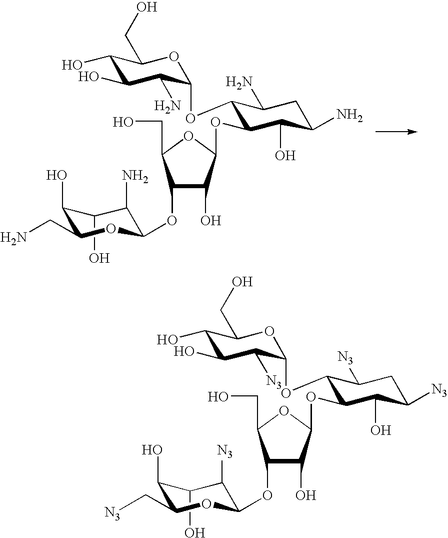

The exocyclic amino groups of Paromomycin were converted into the corresponding azido groups according to the procedure of Wong (Greenberg, W. A.; Priestley, E. S.; Sears, P. S.; Alper, P. B.; Rosenbohm, C. et al. Design and Synthesis of New Aminoglycoside Antibiotics Containing Neamine as an Optimal Core Structure: Correlation of Antibiotic Activity with in Vitro Inhibition of Translation. J. Am. Chem. Soc. 1999, 121, 6527-6541) using paromomycin instead of neomycin.

¹H NMR (300 MHz, DMSO) δ 1.36 (q, J=12 Hz, 1H), δ 1.99-2.06 (m, 1H) δ 3.37-3.73 (m, 1H) δ 2.97-3.02 (m, 1H), δ 3.19-3.27 (m, 1H), δ 3.37-3.73 (m, 15H), δ 3.88-3.95 (m, 2H), δ 4.16-4.25 (m, 2H), δ 4.44 (t, J=5.7 Hz, 1H) δ 4.75 (t, J=4.8 Hz, 1H), δ 4.93 (d, J=5.2 Hz, 1H), δ 5.03 (d, J=1.6 Hz, 1H), δ 5.15 (d, J=5.1 Hz, 1H) δ 5.22 (d, J=4.6 Hz, 1H), δ 5.28 (s, 1H), δ 5.39 (d, J=5.7 Hz, 1H), δ 5.59 (t, J=4.8 Hz, 2H), δ 5.67 (d, J=3.7 Hz, 1H); 13C NMR δ 106.97, 97.64, 95.89, 83.22, 81.67, 75.60, 74.66, 74.13, 72.98, 72.80, 70.30, 70.00, 69.81, 66.99, 63.02 61.50, 60.40, 59.85, 59.66, 59.21, 50.77, 31.46 LCMS m/z 768.0 (M+Na), (>99% purity).

### Example 2

### Selective protection of the 6′-position with Tips

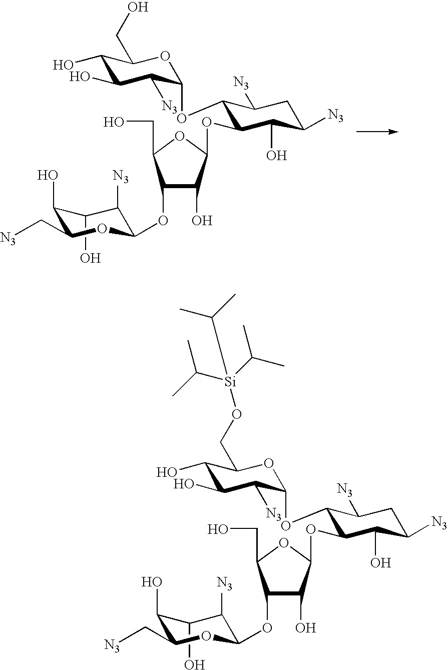

To an oven dried 50.0 mL bottom flask equipped with magnetic stirrer was added per-azidoparomomycin from the above reaction (2.63 g, 3.5 mmol), 4-DMAP (1.25 g, 10.2 mmol) and anhydrous DMF (28.0 mL). The resulting clear solution was cooled to 0 C in ice-bath while stirring under nitrogen. Triisopropylsilylchloride (0.89 ml, 42.3 mmol) was added dropwise to the stirred reaction mixture via syringe. The reaction was continued stirred for two hours maintaining the temperature at 0° C. The reaction mixture was then partitioned between ethyl acetate and 10% aqueous NaHCO₃ solution. The organic layer was separated and washed with saturated brine solution and dried over Na₂SO₄, filtered and evaporated to dryness to afforded clear oil. The product was obtained after purification by flash chromatography (1.57 g, 50% yield) using gradients of CHCl₃/MeOH (97:3).

1H NMR (300 MHz, DMSO) δ 1.36 (q, J=12 Hz, 1H), δ 1.90-1.22 (m, 21H) δ 2.06-2.10 (m, 1H) δ 2.97-3.03 (m, 7H), δ 3.08-3.98 (m, 13H), δ 4.15 (s, 2H), δ 4.6 (t, J=60.4 Hz, 1H), δ 4.94 (d, J=5.0 Hz, 1H), δ 4.99-5.03 (m, 1H), δ 5.14 (d, J=3.73 Hz, 1H), δ 5.20 (d, J=4.6 Hz, 1H), δ 5.27 (s, 1H), δ 5.44 (d, J=5.5 Hz, 1H), δ 5.59 (d, J=3.90 Hz, 1H), δ 5.68 (d, J=6.2 1H), δ 5.79 (d, J=3.73 Hz, 1H), δ 6.62 (dd, J=5.09, 1.5 Hz, 2H), 68.10 (d, J=6.56 Hz, 1H); 13C NMR δ 154.19, 148.11, 108.18, 106.63, 97.56, 95.38, 83.08, 81.62, 75.87, 75.52, 74.00, 73.79, 73.10, 72.76, 70.41, 70.26, 69.76, 66.96, 63.31, 62.91, 62.20, 59.79, 59.58, 59.15, 50.77, 38.64, 31.72, 17.81, 17.79, 11.37, 0.00 LCMS m/z 924 (M+Na), (>99% purity).

### Example 3

### Benzyl Protection of Hydroxyl Groups

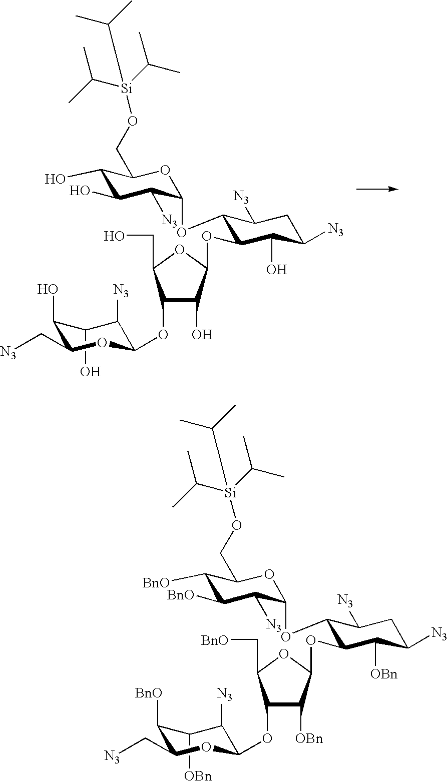

To a 50.0 mL bottom flask equipped with magnetic stirrer was added the tips protected compound from the previous example (3.77 g, 4.18 mmol) dissolved in anhydrous DMF (20.0 mL). The resulting clear solution was cooled to 0° C. in ice-bath while stirring under nitrogen. 60% NaH (2.34 g, 58.5 mmol) was then added slowly and stirred for 20 minutes. BnBr (4.97 mL, 41.87 mmol) was added dropwise to the stirred reaction mixture via syringe. Temperature of 0° C. was maintained for 1 h followed by 3 h at room temperature. The reaction was then cooled at 0° C. and quenched with saturated NaHCO₃ solution (2.0 mL) dropwise. The reaction mixture was then partitioned between DCM and 10% aqueous NaHCO₃ solution. The organic layer was separated and washed with saturated brine solution and dried over Na₂SO₄, filter and evaporated to dryness to afforded clear oil which was purified by silica gel chromatography using gradients of Hexane/EtOA (9:1) to afford the title compound (6.02 g, 93% yield) which was used as is in the next step.

### Example 4

### Selective deprotection of the 6′-position of perbenzylated 6′-O-Tips-perazidoparomomycin and oxidation to the aldehyde

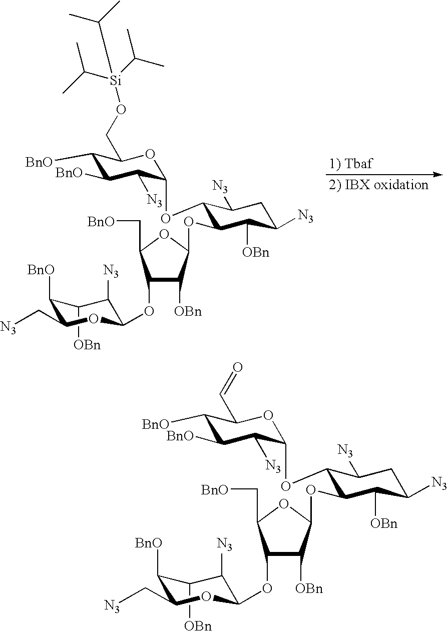

To a 50.0 mL bottom flask equipped with magnetic stirrer was added the benzyl protected 6′-O-Tips-perazidoparomomycin (6.0 g, 3.92 mmol) dissolved in anhydrous THF (20 mL). The resulting clear solution was cooled to 0° C. in ice-bath while stirring under nitrogen. 1.0M TBAF.THF (8.63 mL, 7.84 mmol) was added dropwise to the stirred reaction mixture via syringe and the reaction was then allowed to proceed at room temperature. The reaction was quenched with saturated NH₄CO₃ solution (30.0 mL), extracted with EtOAc and evaporated to dryness to afforded the product as a yellow oil which could be purified by silica gel chromatography using gradients of Hexane/EtOA (8:2) to afforded the title compound (5.4 g, 83% yield) as a white foam. This product (470 mg) was treated with IBX in DMSO (1.2 mL) and THF (1.0 mL) at room temperature for 2.5 hours. At that time, DCM (15 mL) and H2O (10 mL) were added and the aqueous layer was separated and extracted twice more (15 mL). The combined organic layers were dried (Na2SO4), filtered and evaporated to give crude product which could be purified by silica gel chromatography using gradients of Hexane/EtOA (7:3) to afford the title compound (409 mg, 50% yield).

### Example 5

### General procedure for reductive amination and deprotection

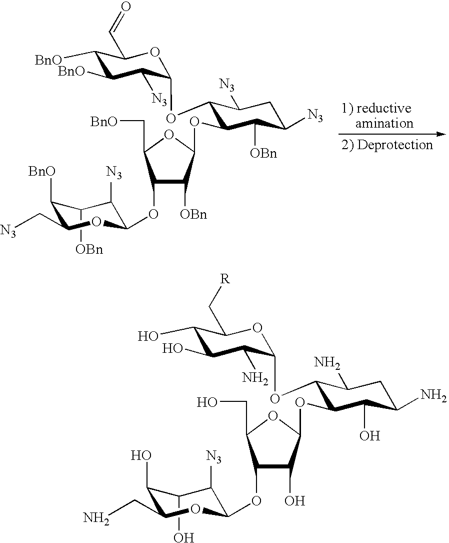

The crude aldehyde (36 umoles) was dissolved in dry MeOH (2 mL) and dry THF (1 mL). To this solution was added the appropriate amine (5 equivalents) in MeOH (2 mL) with the pH adjusted to 5 with AcOH. NaCNBH₃ (4 equiv) was then added and the mixture was allowed to stir for 16 h, at which time the reaction was quenched with NaHCO₃. The reaction was evaporated to dryness, and then the crude mixture was partitioned between DCM and 10% aqueous NaHCO₃ solution. The organic layer was separated and washed with saturated brine solution and dried over Na₂SO₄, filter and evaporated to dryness to afforded clear oil which was purified by silica gel chromatography using gradients of DCM:MeOH (96:4) to afford the protected amine, which was used as is in the next step. To the protected amine was added 2 mL of EtOH, Raney nickel (25-50 mg) and hydrazine (7-14 equivalents). After the reaction had gone to completion as determined by LCMS, the reaction was filtered and evaporated to give the crude perbenzylated product. This was treated with hydrogen (1 atm), palladium (II) hydroxide (2.5 mg) in AcOH (1 mL) and THF (1 mL) to give, after 24 hours, the title compound after lyophilization.

### Example 6

### Preparation of IBIS00561085

Using 4M dimethylamine in methanol in the general procedure above gave the title compound. LCMS m/z 643 (M+H), (>95% purity). ¹H NMR was consistent with the structure.

### Example 7

### Preparation of IBIS00561096

Using 1,3-diaminopropane in the general procedure above gave the title compound. LCMS m/z 672 (M+H), (>95% purity). ¹H NMR was consistent with the structure.

### Example 8

### Preparation of IBIS00561142

Using morpholine in the general procedure above gave the title compound. LCMS m/z 685 (M+H), (>95% purity). ¹H NMR was consistent with the structure.

### Example 9

### Preparation of IBIS00561082

Using N-Boc-hydrazine in the general procedure above gave the title compound. LCMS m/z 730 (M+H), (>95% purity). ¹H NMR was consistent with the structure.

### Example 10

### Preparation of IBIS00561086

Using 2.0 M methylamine in methanol in the general procedure above gave the title compound. LCMS m/z 629 (M+H), (>95% purity). ¹H NMR was consistent with the structure.

### Example 11

### Preparation of IBIS00561140

Using 1,4-butylamine in the general procedure above gave the title compound. LCMS m/z 686 (M+H), (>95% purity). ¹H NMR was consistent with the structure.

### Example 12

### Preparation of IBIS00561930

Using p-Methylphenethylamine in the general procedure above gave the title compound. LCMS m/z 733 (M+H), (>95% purity). ¹H NMR was consistent with the structure.

### Example 13

### Preparation of IBIS00561095

Using isopropylamine in the general procedure above gave the title compound. LCMS m/z 657 (M+H), (>95% purity). ¹H NMR was consistent with the structure.

### Example 14

### Preparation of IBIS561087

Using hydrazine in the general procedure above gave the title compound. LCMS m/z 630 (M+H), (>95% purity). ¹H NMR was consistent with the structure.

### Example 15

### Preparation of IBIS00561141

Using phenethylamine in the general procedure above gave the title compound. LCMS m/z 719 (M+H), (>95% purity). ¹H NMR was consistent with the structure.

### Example 16

### Preparation of IBIS00561931

Using N-methyl-2-phenethylamine in the general procedure above gave the title compound. LCMS m/z 733 (M+H), (>95% purity). ¹H NMR was consistent with the structure.

### Example 17

### Preparation of IBIS00561932

Using phenpropylamine in the general procedure above gave the title compound. LCMS m/z 733 (M+H), (>95% purity). ¹H NMR was consistent with the structure.

### Example 18

### Preparation of IBIS00561938

Using 4-(phenyl)-2-phenethylamine in the general procedure above gave the title compound. LCMS m/z 801 (M+H), (>95% purity). ¹H NMR was consistent with the structure.

### Example 19

### Preparation of IBIS00561935

Using o-methoxyphenethylamine in the general procedure above gave the title compound. LCMS m/z 749 (M+H), (>95% purity). ¹H NMR was consistent with the structure.

### Example 20

### Preparation of IBIS00561944

Using p-Fluorophenethylamine in the general procedure above gave the title compound. LCMS m/z 737 (M+H), (>95% purity). ¹H NMR was consistent with the structure.

### Example 21

### Preparation of IBIS00561936

Using β-methylphenethylamine in the general procedure above gave the title compound. LCMS m/z 733 (M+H), (>95% purity). ¹H NMR was consistent with the structure.

### Example 22

### Preparation of IBIS00561942

Using p-(Trifluoromethyl)phenethylamine in the general procedure above gave the title compound. LCMS m/z 787 (M+H), (>95% purity). ¹H NMR was consistent with the structure.

### Example 23

### Preparation of IBIS00561933

Using p-methoxyphenethylamine in the general procedure above gave the title compound. LCMS m/z 749 (M+H), (>95% purity). ¹H NMR was consistent with the structure.

### Example 24

### Preparation of IBIS00561939

Using indoline in the general procedure above gave the title compound. LCMS m/z 723 (M+H), (>95% purity). ¹H NMR was consistent with the structure.

### Example 25

### Preparation of IBIS00561937

Using β-hydroxyphenethylamine in the general procedure above gave the title compound. LCMS m/z 749 (M+H), (>95% purity). ¹H NMR was consistent with the structure.

### Example 26

### Preparation of IBIS00561943

Using m-(Trifluoromethyl)phenethylamine in the general procedure above gave the title compound. LCMS m/z 787 (M+H), (>95% purity). ¹H NMR was consistent with the structure.

### Example 27

### Preparation of IBIS00561934

Using m-methoxyphenethylamine in the general procedure above gave the title compound. LCMS m/z 749 (M+H), (>95% purity). ¹H NMR was consistent with the structure.

### Example 28

### Preparation of IBIS00561940

Using tryptamine in the general procedure above gave the title compound. LCMS m/z 766 (M+H), (>95% purity). ¹H NMR was consistent with the structure.

### Example 29

### Preparation of IBIS00561941

Using 1-napthylethylamine in the general procedure above gave the title compound. LCMS m/z 773 (M+H), (>95% purity). ¹H NMR was consistent with the structure.

### Example 30

### Preparation of IBIS00561947

Using 4-(aminoethyl)pyridine in the general procedure above gave the title compound. LCMS m/z 726 (M+H), (>95% purity). ¹H NMR was consistent with the structure.

### Example 31

### Preparation of IBIS00561945

Using 3-(aminoethyl)pyridine in the general procedure above gave the title compound. LCMS m/z 726 (M+H), (>95% purity). ¹H NMR was consistent with the structure.

### Example 32

### Preparation of IBIS00561946

Using 2-(aminoethyl)pyridine in the general procedure above gave the title compound. LCMS m/z 726 (M+H), (>95% purity). ¹H NMR was consistent with the structure.

### Example 33

### Staphylococcus aureus (Smith strain ATCC 13709) MOUSE PROTECTION ASSAY

Two of the novel aminoglycoside compounds of the invention were examined for their anitbacterial activity against staphylococcus aureus. The mice were infected IP with 0.5 ml 10 staphylococcus aureus in 10% mucin. There were 10 mice in each treated group with treatments at 1 hour and 3 hour post infection. The Ibis compounds (IBIS00561085 R₄═R₅═CH₃ and IBIS00561141 R₄═H, R₅═(CH₂)₂C₆H₅) were used at 75 mg/kg, 37.5 mg/kg, 18.8 mg/kg, 9.4 mg/kg, 4.7 mg/kg, 2.3 mg/kg, 1.17 mg/kg and 0.5 mg/kg. Amakacin, Paromomycin and Neomycin were used as the positive controls at concentration of 2 mg/kg, 1 mg/kg and 0.5 mg/kg.

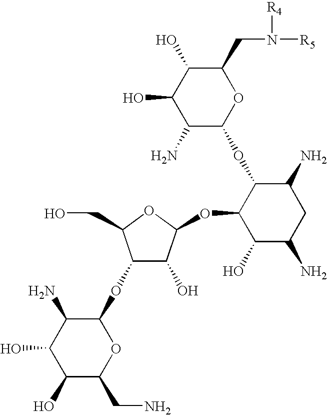

| Staph | | |
|---|---|---|
| Conc. | Antibiotic conc. | # Dead mice/Total mice in group |
| 10⁹ | 0 | 5/5 (10% Mucin) |
| 10⁸ | 0 | 5/5 (10% Mucin) |
| 10⁷ | 0 | 1/5 (10% Mucin) |
| 10⁶ | 0 | 0/5 (10% Mucin) |
| 0 | 0 | 0/10 (10% Mucin) |
| 10⁶ | 0 | 9/10 (10% Mucin) |
| 10⁷ | 0 | 9/10 (10% Mucin) |
| 10⁶ | Amikacin 2 mg/kg | 8/10 (10% Mucin) |
| 10⁶ | Amikacin 1 mg/kg | 10/10 (10% Mucin) |
| 10⁶ | Amikacin 0.5 mg/kg | 8/10 (10% Mucin) |
| 10⁶ | Paromomycin 2 mg/kg | 9/10 (10% Mucin) |
| 10⁶ | Paromomycin 1 mg/kg | 10/10 (10% Mucin) |
| 10⁶ | Paromomycin 0.5 mg/kg | 10/10 (10% Mucin) |
| 10⁶ | Neomycin 2 mg/kg | 4/10 (10% Mucin) |
| 10⁶ | Neomycin 1 mg/kg | 10/10 (10% Mucin) |
| 10⁶ | Neomycin 0.5 mg/kg | 7/10 (10% Mucin) |
| 10⁶ | 00561085 75 mg/kg | 0/10 (10% Mucin) |
| 10⁶ | 00561085 37 mg/kg | 0/10 (10% Mucin) |
| 10⁶ | 00561085 18 mg/kg | 0/10 (10% Mucin) |
| 10⁶ | 00561085 9 mg/kg | 0/10 (10% Mucin) |
| 10⁶ | 00561085 4.5 mg/kg | 1/10 (10% Mucin) |
| 10⁶ | 00561085 2 mg/kg | 7/10 (10% Mucin) |
| 10⁶ | 00561085 1 mg/kg | 7/10 (10% Mucin) |
| 10⁶ | 00561085 0.5 mg/kg | 8/10 (10% Mucin) |
| 10⁶ | 00561141 75 mg/kg | 0/10 (10% Mucin) |
| 10⁶ | 00561141 37 mg/kg | 0/10 (10% Mucin) |
| 10⁶ | 00561141 18 mg/kg | 0/10 (10% Mucin) |
| 10⁶ | 00561141 9 mg/kg | 0/10 (10% Mucin) |
| 10⁶ | 00561141 4.5 mg/kg | 0/10 (10% Mucin) |
| 10⁶ | 00561141 2 mg/kg | 0/10 (10% Mucin) |
| 10⁶ | 00561141 1 mg/kg | 0/10 (10% Mucin) |
| 10⁶ | 00561141 0.5 mg/kg | 7/10 (10% Mucin). |

### Example 34

### General Assay Protocols

### Measurement of 16S Kd by FTICR Mass Spectrometry

Was performed according to the published report; Griffey, R. H.; Sannes-Lowery, K. A.; Drader, J. J.; Mohan, V.; Swayze, E. E. et al. Characterization of Low Affinity Complexes Between RNA and Small Molecules Using Electrospray Ionization Mass Spectrometry. J. Am. Chem. Soc. 2000, 122, 9933-9938.

### Coupled Bacterial Transcription/Translation Assay (T/T)

The DNA template, pBest Luc™ (Promega), is a plasmid containing a reporter gene for firefly luciferase fused to a strong tac promoter and ribosome binding site. Messenger RNA from 1 μg pBestLuc was transcribed and translated in E. coli S30 bacterial extract in the presence or absence of test compound. Compounds were tested in a black 96 well microtiter plate with an assay volume of 35 mL. Each test well contained: 5 μL test compound, 13 μL S30 premix (Promega), 4 μL 10× complete amino acid mix (1 mM each), 5 μL E. coli S30 extract and 8 μL of 0.125 μg/μL pBest Luc™. The transcription/translation reaction was incubated for 35 minutes at 37° C. followed by detection of functional luciferase with the addition of 30 μL LucLite™ (Packard). Light output was quantitated on a Packard TopCount.

### Minimum Inhibitory Concentrations (MIC)

The assays are carried out in 150 μL volume in duplicate in 96-well clear flat-bottom plates. The bacterial suspension from an overnight culture growth in appropriate medium is added to a solution of test compound in 2.5% DMSO in water. Final bacterial inoculum is approximately 10²-10³ CFU/well. The percentage growth of the bacteria in test wells relative to that observed for a control wells containing no compound is determined by measuring absorbance at 595 nm (A₅₉₅) after 20-24 hours at 37° C. The MIC is determined as a range of concentration where complete inhibition of growth is observed at the higher concentration and bacterial cells are viable at the lower concentration. Both ampicillin and tetracycline are used as antibiotic positive controls in each screening assay.

Selected compounds were assayed using the protocols listed herein and the results are tabulated below.

| | 18S | 16S | Trans/Trans | MIC (uM) | |
|---|---|---|---|---|---|
| IBIS# | Kd (uM) | Kd (uM) | IC50 (uM) | E. Coli. | S. Aureus |
| IBIS00561082 | 1.351 | 0.0149 | 0.308 | 6-12 | 12-25 |
| IBIS00560185 | 0.6705 | 0.552 | 0.273 | 3-6 | 2-3 |
| IBIS00560186 | 1.161 | 0.3624 | 0.159 | 2-3 | 2-3 |
| IBIS00560187 | 1.662 | 0.5641 | 0.235 | 12-25 | 3-6 |
| IBIS00561095 | 1.205 | 1.178 | 0.2022 | 2.5-5.0 | 1.25-2.5 |
| IBIS00561096 | 1.449 | 1.592 | 0.1753 | 2.5-5.0 | 1.25-2.5 |
| IBIS00561140 | 6.023 | 6.265 | 0.032 | 2.5-5.0 | 10.0-20.0 |
| IBIS00561141 | 2.157 | 0.4962 | 0.200 | 1.25-2.5 | 0.3125-0.625 |
| IBIS00561142 | 3.005 | 3.639 | 0.349 | 20.0-40.0 | 10.0-20.0 |
| IBIS00561930 | NT | NT | NT | 2.5-5.0 | 0.6-1.2 |
| IBIS00561931 | NT | NT | NT | 2.5-5.0 | 0.31-0.62 |
| IBIS00561932 | NT | NT | NT | 0.6-1.2 | 0.31-0.62 |
| IBIS00561933 | NT | NT | NT | 1.25-2.5 | 0.31-0.62 |
| IBIS00561934 | NT | NT | NT | 1.25-2.5 | 0.31-0.62 |
| IBIS00561935 | NT | NT | NT | 1.25-2.5 | 0.31-0.62 |
| IBIS00561936 | NT | NT | NT | 1.25-2.5 | 0.16-0.31 |
| IBIS00561937 | NT | NT | NT | 5.0-10.0 | 0.6-1.2 |
| IBIS00561938 | NT | NT | NT | 1.25-2.5 | 0.31-0.62 |
| IBIS00561939 | NT | NT | NT | 1.25-2.5 | 0.31-0.62 |
| IBIS00561940 | NT | NT | NT | 1.25-2.5 | 1.25-2.5 |
| IBIS00561941 | NT | NT | NT | 1.25-2.5 | 0.31-0.62 |
| IBIS00561942 | NT | NT | NT | 1.25-2.5 | 0.31-0.62 |
| IBIS00561943 | NT | NT | NT | 0.6-1.2 | 0.16-0.31 |
| IBIS00561944 | NT | NT | NT | 0.6-1.2 | 0.16-0.31 |
| IBIS00561945 | NT | NT | NT | 1.25-2.5 | 0.31-0.62 |
| IBIS00561946 | NT | NT | NT | 1.25-2.5 | 0.31-0.62 |
| IBIS00561947 | NT | NT | NT | 1.25-2.5 | 0.31-0.62 |

### Example 35

### Mass Spectrometry Based Binding Assay

Screening is performed by measuring the formation of non-covalent complexes between a single ligand or ligand mixture and the appropriate RNA target, along with suitable control structured RNA target(s) simultaneously using a 9.4 T FT-ICR mass spectrometer as detector. Full experimental details of the assay have been described in related literature (Sannes-Lowery, et al. in TrAC, Trends Anal. Chem. 2000, 19, 481-491 and Sannes-Lowery, et al. in Anal. Biochem. 2000, 280, 264-271. In a typical experiment, 10:L of an aqueous solution containing 100 mM ammonium acetate buffer, 2.5 or 5:M of each RNA, and 33% isopropyl alcohol (to aid ion desolvation) is prepared with different concentrations of each ligand or ligand mixture. Samples are introduced into the electrospray ionization source (negative ionization mode) at 1:L/min and ions are stored for 1 sec in an RF-only hexapole following desolvation. The abundances were integrated from the respective ions for free RNA and the ligand-RNA complex. The primary (1:1 RNA:ligand) and secondary (1:2 complex, if observed) KD values are determined by titrating a single ligand through a concentration range of 0.25-25 μM with an RNA target concentration of 0.10 μM. The peak ratios are measured at each concentration, then a plot of complex/free RNA versus concentration of ligand added is fitted to a second (or higher) order binding polynomial to determine the KD.

Each reference cited herein, including but not limited to, patents, patent applications, patent publications, articles, treatises, and texts, is hereby incorporated by reference in its entirety.

### Listing of Compounds

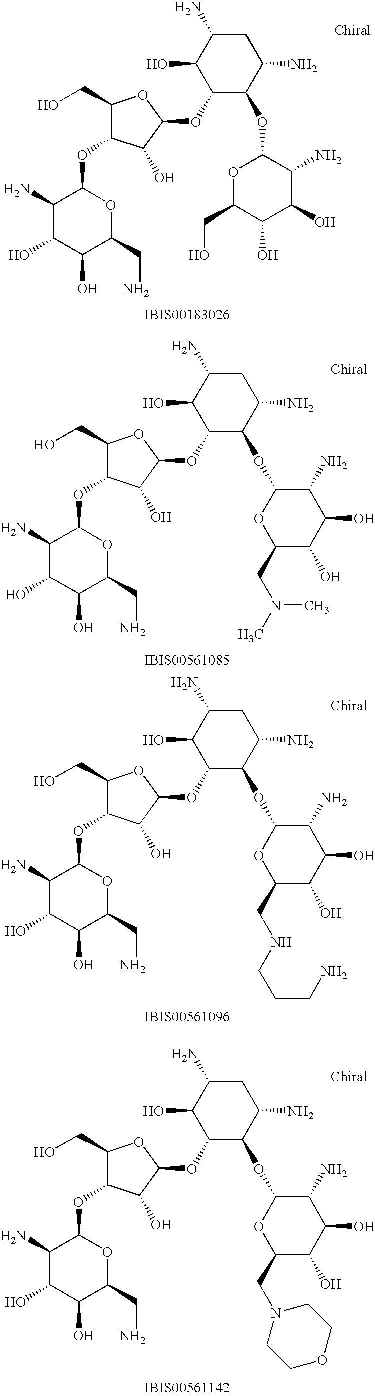

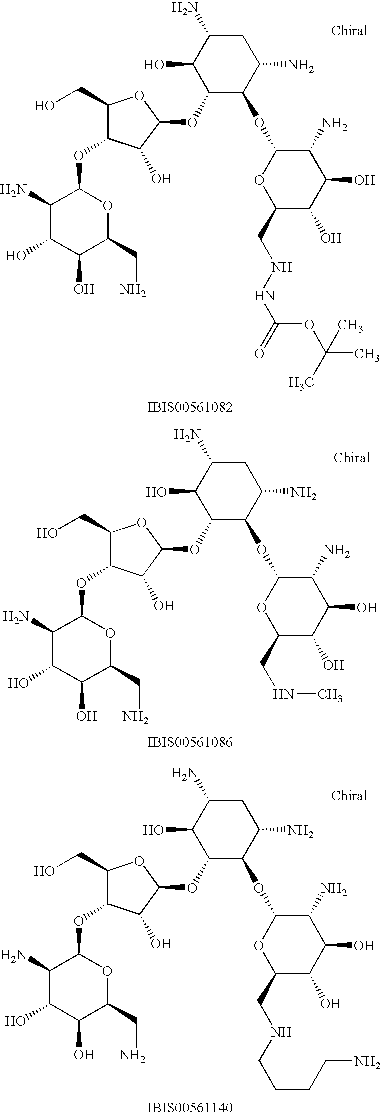

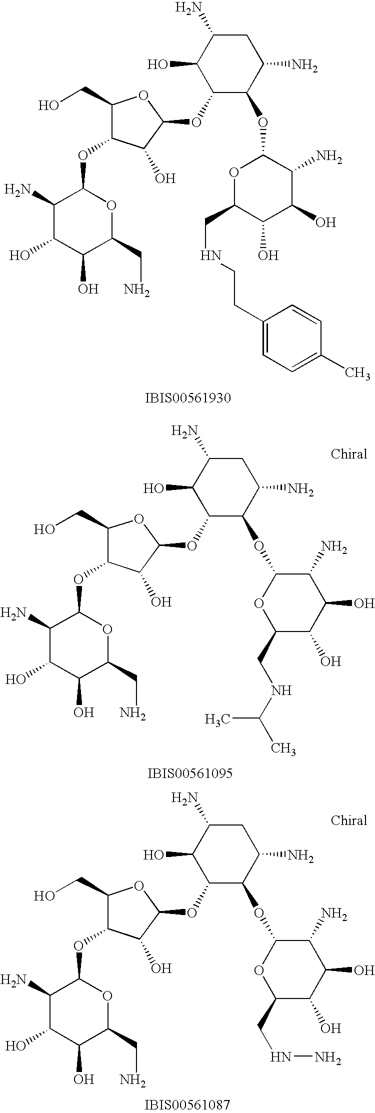

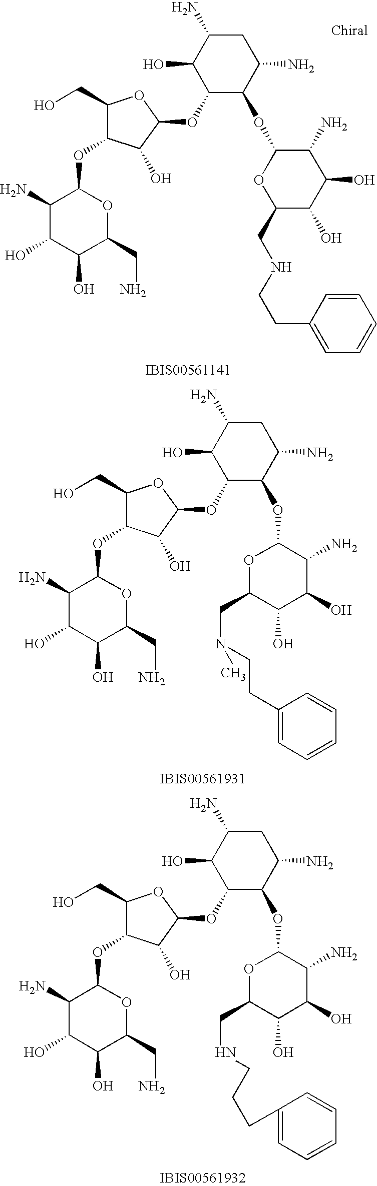

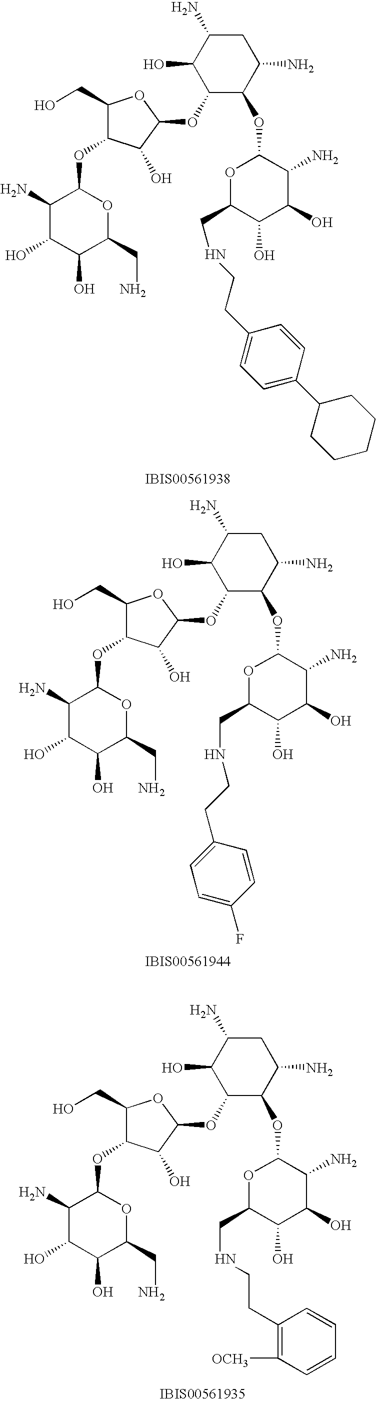

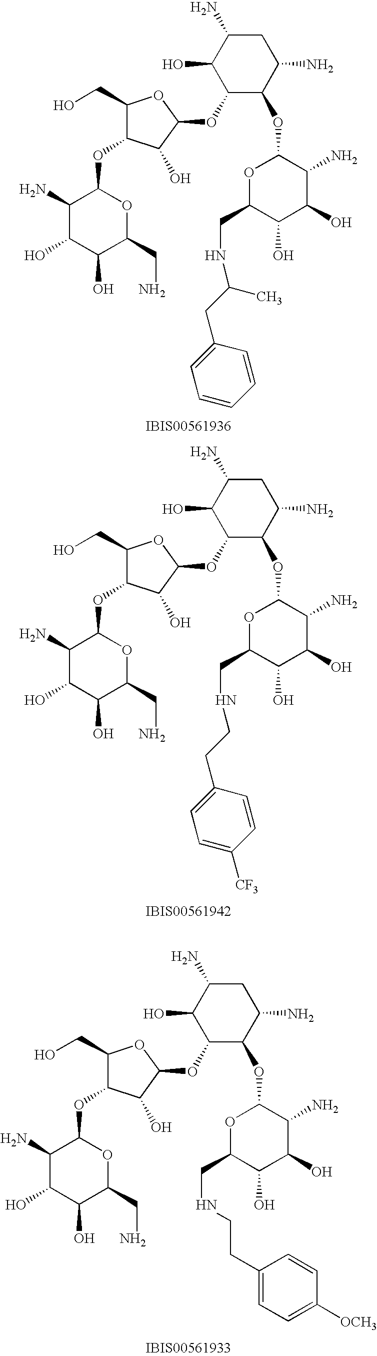

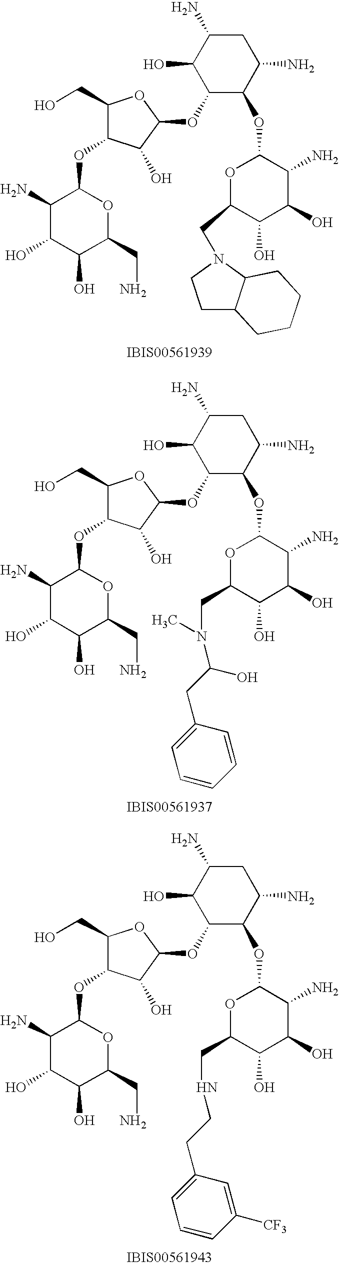

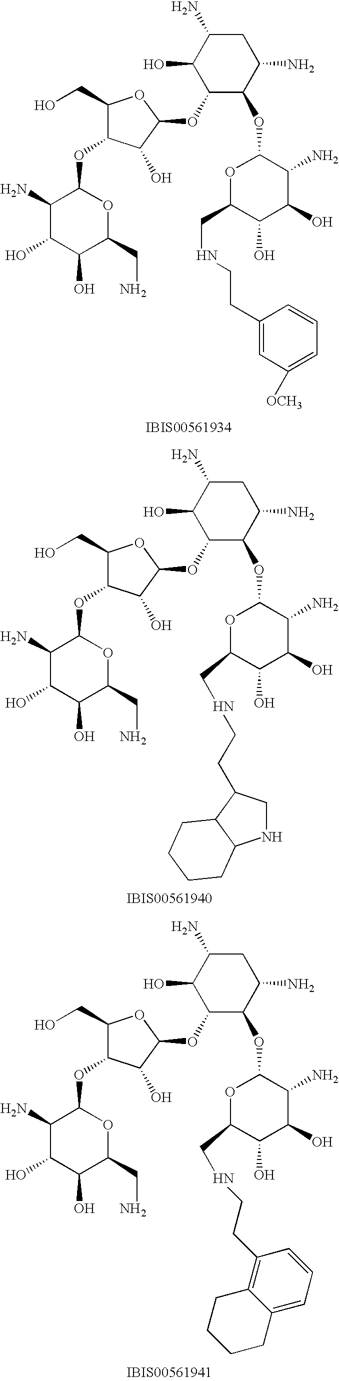

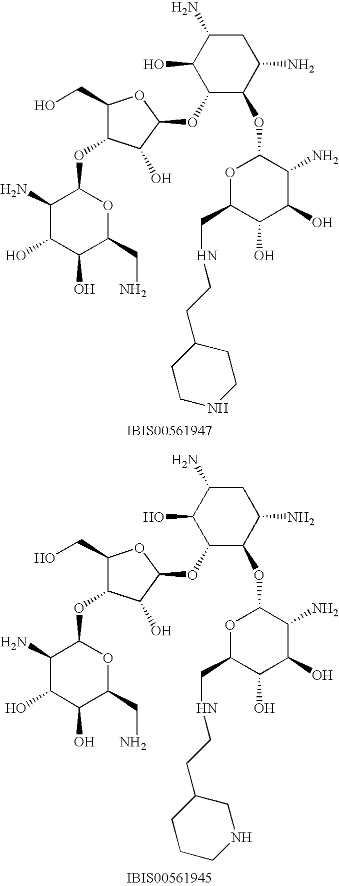

What is claimed is:

1. A compound of formula I: 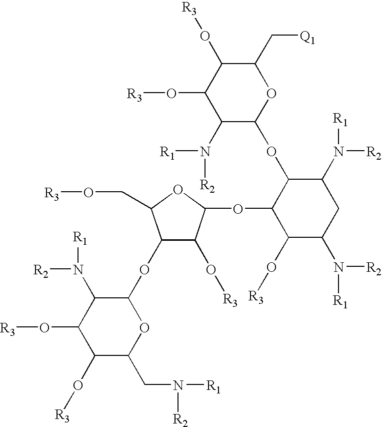
wherein:
each R₁ and R₂ is, independently, H or an amino protecting group;
each R₃ is, independently, H or a hydroxyl protecting group;
Q₁ is a NR₄R₅, a protected amino group or a nitrogen containing heterocycle wherein said heterocycle is covalently linked to said compound through the nitrogen atom;
R₄ is H, an amino protecting group, C₁-C₁₂ alkyl or substituted C₁-C₁₂ alkyl;
R₅ is amino, substituted amino, an amino protecting group, hydroxy, C₁-C₁₂ alkyl, substituted C₁-C₁₂ alkyl, C₂-C₁₂ alkenyl, substituted C₂-C₁₂ alkenyl, C₂-C₁₂ alkynyl or substituted C₂-C₁₂ alkynyl;
wherein said substituted groups are mono or poly substituted with optionally protected substituent groups each independently selected from halogen, OJ₃, NJ₁J₂, C(═O)—NJ₁J₂, N(H)C(═O)-J₁, N(J₁)-(CH₂)ₙ—OJ₃, N(J₁)-(CH₂)ₙ—NJ₁J₂, C₅-C₂₀ aryl, substituted C₅-C₂₀ aryl, C₅-C₇ alicyclic radical, substituted C₅-C₇ alicyclic radical, heterocycle radical, substituted heterocycle radical, azido, carboxy, acyl (C(═O)—X), ═O, cyano, sulfonyl (S(═O)₂—X) and sulfoxyl (S(═O)—X);
each X is, independently, H, C₁-C₁₂ alkyl or substituted C₁-C₁₂ alkyl;
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—X), substituted acyl, a heterocycle radical or a substituted heterocycle radical;
each J₃ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ aminoalkyl, substituted C₁-C₁₂ aminoalkyl or a hydroxyl protecting group; and
n is from 1 to 20.

2. The compound of claim 1 wherein each R₁ and R₂ is H.

3. The compound of claim 1 wherein each R₃ is H.

4. The compound of claim 1 wherein each of said substituent groups is independently, OH, NH₂, N(H)alkyl, C(═O)—N(H)J₂, N(H)C(═O)-J₁, N(J₁)-(CH₂)ₙ—OJ₃, N(J₁)-(CH₂)ₙ—NJ₁J₂, substituted C₅-C₇ alicyclic radical, C₅-C₇ alicyclic radical, C₅-C₂₀ aryl, substituted C₅-C₂₀ aryl, a heterocycle radical or a substituted heterocycle radical.

5. The compound of claim 1 wherein Q₁ is NR₄R₅.

6. The compound of claim 5 wherein R₄ is C₁-C₁₂ alkyl or substituted C₁-C₁₂ alkyl.

7. The compound of claim 5 wherein R₄ is H.

8. The compound of claim 5 wherein R₅ is NH₂, C₁-C₁₂ alkyl or mono or poly substituted C₁-C₁₂ alkyl.

9. The compound of claim 8 wherein R₅ is mono or poly substituted C₁-C₁₂ alkyl wherein each substituent group is independently selected from halogen, OH, NJ₁J₂, C₅-C₂₀ aryl, substituted C₅-C₂₀ aryl, C₅-C₇ alicyclic radical, substituted C₅-C₇ alicyclic radical, heterocycle radical and substituted heterocycle radical.

10. The compound of claim 9 wherein each of said substituent groups is, independently, NH₂, phenyl, substituted phenyl, heterocycle radical or substituted heterocycle radical.

11. The compound of claim 10 wherein said substituted phenyl comprises at least one substituent group selected from halogen, C₁-C₁₂ alkyl, CF₃, alicyclic radical, OCH₃ and heterocyclic radical.

12. The compound of claim 9 wherein said mono or poly substituted C₁-C₁₂ alkyl comprises at least two substituent groups.

13. The compound of claim 12 wherein said at least two substituent groups are different and are selected from OH, C₁-C₁₂ alkyl, C₅-C₂₀ aryl and substituted C₅-C₂₀ aryl.

14. The compound of claim 1 having the configuration:

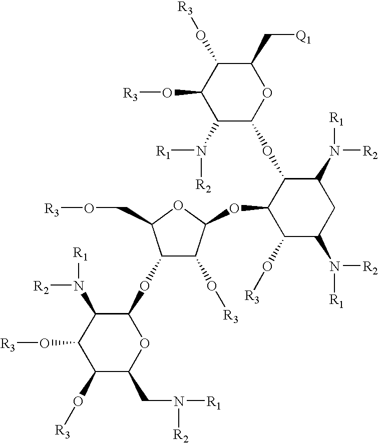

15. The compound of claim 1 for use in therapy.
